(19) United States
(12) Patent Application Publication
(10) Pub. No.: US 2014/0323862 A1
(43) Pub. Date: Oct. 30, 2014

(54) SYSTEM AND METHODS FOR DETERMINING TISSUE ELASTICITY

(71) Applicant: The Trustees of Columbia University in the City of New York, New York, NY (US)

(72) Inventors: Ronald Silverman, West Nyack, NY (US); Raksha Urs, Teaneck, NJ (US)

(21) Appl. No.: 14/326,168

(22) Filed: Jul. 8, 2014

Related U.S. Application Data
(63) Continuation of application No. PCT/US2013/020764, filed on Jan. 9, 2013.
(60) Provisional application No. 61/584,519, filed on Jan. 9, 2012.
(60) Provisional application No. 61/588,033, filed on Jan. 18, 2012.

(51) Int. Cl. A61B 8/08; A61B 8/10; A61B 5/00

(52) U.S. Cl. CPC A61B 8/485; A61B 5/442; A61B 8/10
USPC 600/438

(57) ABSTRACT

The present disclosed subject matter is directed to medical devices and methods that assist in non-invasively determining elastic properties (e.g., elasticity, viscosity, etc.) of superficial tissues, especially superficial corneal tissues such as the epithelium and stroma, using acoustic energy.

### CROSS REFERENCE TO RELATED APPLICATIONS

This application claims priority to U.S. provisional Patent Application Nos. 61/588,033 filed Jan. 18, 2012 and 61/584,519, filed Jan. 9, 2012, the disclosures of which are hereby incorporated by reference in their entirety.

### FIELD

The disclosed subject matter relates to a medical device for determining tissue elasticity and methods for their use.

### BACKGROUND

A variety of non-invasive elastographic methods and devices are known for determining elasticity of corneal tissue. Typically, such methods use mechanical compression or tonometry (e.g., air-puff) techniques. One drawback from these known methods and devices is that they suffer from low resolution, and consequently, for example, are unable to accurately resolve the properties of the individual corneal layers, namely the epithelium and stroma. Because of these drawbacks conventional techniques are not suitable for assessing and treating diseases corresponding to the elastic properties of only one of the tissues. For example, in assessing and treating keratoconus, it is desired to determine the elastic properties of the corneal stroma apart from other corneal tissues. Correspondingly, an accurate measurement of corneal elasticity is a prerequisite for an accurate measurement of intra-ocular pressure, important in determination of a patient's risk for glaucoma. Current non-invasive methods of determining elastic properties of tissues are not suitable for determining such properties of the corneal stroma alone due to its proximity to the corneal epithelium. Therefore, either the elastic properties of the epithelium and stroma are conflated or it is necessary to scrape away the corneal epithelium to determine the elastic properties of the stroma alone.

There thus remains a need for an efficient and economic method and system for non-invasively measuring the elastic properties of tissues with a greater resolution than is available in the art.

### SUMMARY

In one aspect, a medical device is provided for measuring properties (e.g., viscosity, elasticity, cross-linking, Young's modulus, etc.) of superficial tissue. The superficial tissue may comprise first and second tissues. The first tissue may be the corneal epithelium. The second tissue may be corneal stroma. The medical device may include an ultrasonic transducer. The ultrasonic transducer has a center frequency. The center frequency may be at least approximately 25 megahertz. Alternatively, the ultrasonic transducer has a center frequency of between about 25 megahertz to about 50 megahertz. The transducer may be a single element transducer. The medical device also may include a signal generator for producing waveforms and an amplifier. The signal generator may be an arbitrary waveform generator. The waveform generator may be capable of interleaving various types of pulses. For example, the waveform generator may interleave pulses having different shapes, frequencies, or periods. The waveform generator may interleave diagnostic pulses with push pulses. The diagnostic pulses may comprise a voltage spike or a monocycle. The push pulses may have a frequency approximately equal to the center frequency. The signal generator, amplifier and transducer may be placed in electronic communication with each other. The transducer may be configured to direct energy toward the tissue in response to waveforms generated by the signal generator. The energy may be produced, e.g., in the form of acoustic waves. In another embodiment the medical device may be used for determining properties of optical tissues, e.g., tissues of the eye. The tissues may include first and second tissue layers of the cornea. The first tissue may be the corneal epithelium. The second tissue may be the corneal stroma. The medical device may include a transducer. The transducer may be configured to emit output acoustic waves in response to an input waveform. The transducer may also be configured to create an output waveform in response to input acoustic waves. The transducer may additionally be configured to emit pulses at a sufficiently high frequency such that a first echo created at a first tissue in response to the output acoustic waves are differentiable from a second echo created at a second tissue in response to the output acoustic waves. It is contemplated that the first tissue may have a thickness of less than 70 micrometers. For example, the corneal epithelium has a thickness of approximately 50 micrometers. The transducer in the medical device may be a component of an acoustic radiation force impulse imaging device. The transducer may be a single transducer. The transducer may have a center frequency of at least approximately 25 megahertz. Alternatively the transducer may have a center frequency between approximately 25 megahertz and 50 megahertz. The waveform generator may be capable of interleaving various types of pulses. For example, the waveform generator may interleave pulses having different shapes, frequencies, or periods. The waveform generator may interleave diagnostic pulses with push pulses. The diagnostic pulses may comprise a voltage spike or a monocycle. The push pulses may have a frequency approximately equal to the center frequency. The signal generator, amplifier and transducer may be placed in electronic communication with each other. The signal generator may be configured to provide a sufficient number of push waveforms to effect a displacement in at least the first tissue layer. Additionally, the signal generator is configured to provide a sufficient number of push waveforms to maintain the effected displacement.

In another aspect, a system is provided which includes e.g., any of the embodiments of the medical device as heretofore described. The system also may include a digitizer, a storage medium, and a second device. The second device may be a computing device. The computing device may be any device having a processor. The digitizer may be configured to transform an acoustic wave into data. The acoustic wave may correspond to an echo. The acoustic wave may be comprised of a combination of a first wave and a second wave, the first wave corresponding to a first echo, the second wave corresponding to a second echo. The combination may be constructive interference, destructive interference, or a combination thereof. The storage medium may be configured to store the data corresponding to the echoes. The processor may be configured to calculate the elastic properties. The second device may be a PC, laptop, portable computing device, Smartphone, or Personal Digital Assistant. In an alternative embodiment, the system may also include a transmitting device, e.g., a transceiver. The transmitting device may be configured to transmit data, e.g., wirelessly, by wireline, or another storage medium, e.g., a compact disc or USB flash drive.

The disclosed subject matter also includes a method for determining biomechanical properties of at least a first tissue. The method includes a step of providing acoustic energy. The acoustic energy may be originated by a transducer. The acoustic energy may be provided at a frequency of at least approximately 25 megahertz. Alternatively, the acoustic energy may be provided at a frequency of between approximately 25 megahertz and approximately 50 megahertz. The transducer may be driven with a series of diagnostic pulses interleaved with a series of push pulses. The push pulses may have a frequency of at least approximately 25 megahertz. Alternatively, the push pulses may have a frequency of between approximately 25 megahertz and approximately 50 megahertz. The transducer may produce diagnostic acoustic wave from the diagnostic pulses and the transducer may produce push acoustic waves from the push pulses. The method also includes a step of directing the energy toward the first tissue. Response pulses may be generated. The response pulses may be generated from echo waves received at the transducer. The echo waves may be created from acoustic energy reflecting from the first tissue. A property of the first tissue may be determined from the response pulses. In another embodiment, the method may further include generating echo waves from a second tissue. These waves may be received at the transducer. Response pulses may additionally be generated from the echo waves from the second tissue. The first tissue may be corneal epithelium. The second tissue may be corneal stroma. In an alternative embodiment, the response pulses may be digitized as data. This data may be stored. In some embodiments, the viscosity of the first tissue and the viscosity of the second tissue may be calculated. In some embodiments, the elasticity of the first tissue and the elasticity of the second tissue may be calculated. In some embodiments, the cross-linking in the second tissue may be assessed, where cross-linking refers to chemically-induced (e.g., riboflavin+ultraviolet light) modification of the stromal collagen for the purpose of increasing corneal stiffness.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

FIG. 1 is a schematic representation of an embodiment of a medical device for determining tissue elasticity in accordance with the disclosed subject matter.

FIGS. 2A-B are plots of corneal strain as a function of time during and after exposure to ultrasound radiation force.

FIG. 3a is a plot of corneal thickness change during and after exposure of the cornea to acoustic radiation force.

FIG. 3b is a plot of corneal thickness change during the push, with first and second order exponential best fit to the data.

FIG. 4 is an ultrasound image of one line-of-sight through the cornea as a function of time.

FIG. 5 is an ultrasound image of one line-of-sight through the anterior segment of the eye as a function of time.

FIG. 6 is a graphical representation of an interleaved ARF pulse sequence.

FIG. 7 is images of a choroidal melanoma derived from dual 18 MHz/36 MHz transducers.

FIGS. 8a-b show an M-scan demonstrating tissue displacements at the posterior pole of an ex vivo rabbit eye.

FIG. 9 shows a B-scan of the anterior segment of an ex vivo pig eye.

FIG. 10 shows M-mode data of the effect of interfering beams producing vibration on an agar phantom.

FIGS. 11A-B shows a plot of corneal strain during and after exposure to ultrasound radiation force in control (FIG. 11A) and cross-linked (FIG. 11B) rabbit corneas.

FIG. 12 shows the average change in elasticity measured for four weeks in the control and the cross-linked rabbit corneas.

FIG. 13, top, shows an M-scan of the posterior of the rabbit eye in vivo immediately following a 10 msec ARF burst. The vertical axis represents time and the horizontal axis represents tissue depth. The bottom portion of FIG. 13 represents displacement relative to pre-ARF conditions.

FIG. 14A-B shows a B-scan and M-scan, respectively, of the posterior of a proptosed rabbit eye before and after a 10 msec exposure to acoustic radiation force (initiated at horizontal line through image). The arrow indicates the choroid.

FIG. 15 shows a plot of choroidal backscatter amplitude change relative to pre-exposure levels as a function of time. Intraocular pressure is elevated in proptosed eyes and choroidal ischemia occurs as a result. ARFI appears to produce a transient inflow of blood to the choroid.

FIG. 16 shows a plot of standard deviations within and between triplicate acoustic radiation force exposures.

FIGS. 17A-B show B- and M-scans, respectively, of the posterior of the normally seated rabbit eye, demonstrating pulsatile flow in orbital vessels.

FIG. 18 shows a phase-resolved 18 MHz M-scan demonstrating perfusion of the choroid. The diagonal line indicates the approximate slope of the acoustic phase fronts and corresponds to an axial velocity of 1.3 mm/sec.

FIGS. 19A-B show phase-resolved M-scans demonstrating the effect of 10 msec ARFI exposure (horizontal lines) on two regions of the in vivo rabbit choroid before, during, and after ARFI. While ARFI-induced tissue displacements on the order of 10 microns were observed, in normotensive eyes, there was generally little or no increase in choroidal backscatter as was seen in proptosed eyes.

### DETAILED DESCRIPTION

Keratoconus is an ocular condition in which the corneal stroma undergoes thinning and bulging, causing deterioration in vision. The disease occurs primarily in young adults and may lead ultimately to corneal transplantation. There is a general consensus that stromal elastic properties are altered in keratoconus, but there is a paucity of methods for measuring elasticity in vivo. Rather, the effect of cross-linking on corneal stiffness has been determined using ex vivo methods such as histology, microcomputer-controlled biomaterial-testing devices, gel-electrophoresis, super-sonic shear imaging and atomic force microscopy. Of these methods only the super-sonic imaging technique can be used in vivo. Another instrument that can be used in vivo is the Ocular Response Analyzer (ORA). The ORA produces deflection of the cornea in response to an air-puff. The ORA detects when the cornea is flattened (applanated) as it indents under the air pressure and as it recovers. The only measurements are pressure at the two applanation points, from which ‘hysteresis’ is measured. The ORA thus does not provide any information on the dynamics of pressure-generated displacements.

Acoustic radiation force (ARF), however, enables displacements on the order of tens of microns and many thousands of measurements per second. The subject matter of the present disclosure concerns ARF-based devices, systems and methods that are suitable for studying the dynamics of corneal displacements, specifically of the endothelium and stroma, allowing assessment of corneal stiffness. The disclosed subject matter is particularly suited to individually assessing elastic properties of any set of tissues, even those tissues that are positioned within approximately tens of microns or less from each other. For example, the corneal epithelium and the corneal stroma are separated from each other by approximately 10 microns. The distance from the anterior surface of the corneal epithelium to the anterior surface of the corneal stroma is typically approximately 50 microns to approximately 60 microns.

Acoustic radiation force is generated by an ultrasound beam from transfer of momentum from the beam to the tissue through absorption, scattering and reflection. It can be used to investigate the mechanical properties of soft tissue. In soft tissues, most of the momentum transfer is through absorption. Assuming only linear propagation, radiation force F=2αI/c, where α is the tissue absorption coefficient (m⁻¹), I is temporal average acoustic intensity (W/m²) and c is the speed of sound (m/s). For reflection, based on the reflection coefficient, the reflected power is calculated by doubling it to take into account redirection of the beam. The axial displacement of tissue caused by the cornea's absorption of this radiation force is a function of the magnitude of the applied force and tissue elasticity. The force applied on the cornea represents the stress on the cornea. The thinning on the cornea represents the strain. Viscoelastic property information is encoded into the corneal response to the compressional force delivered by the ultrasound.

The exemplary embodiments in accordance with the disclosed subject matter can be used to determine tissue elasticity, and in particular elasticity of superficial tissue. In addition, the disclosed subject matter is particularly suited for determining elastic properties (e.g., elasticity, viscosity, cross-linking, etc.) of superficial corneal tissues such as the corneal epithelium and corneal stroma.

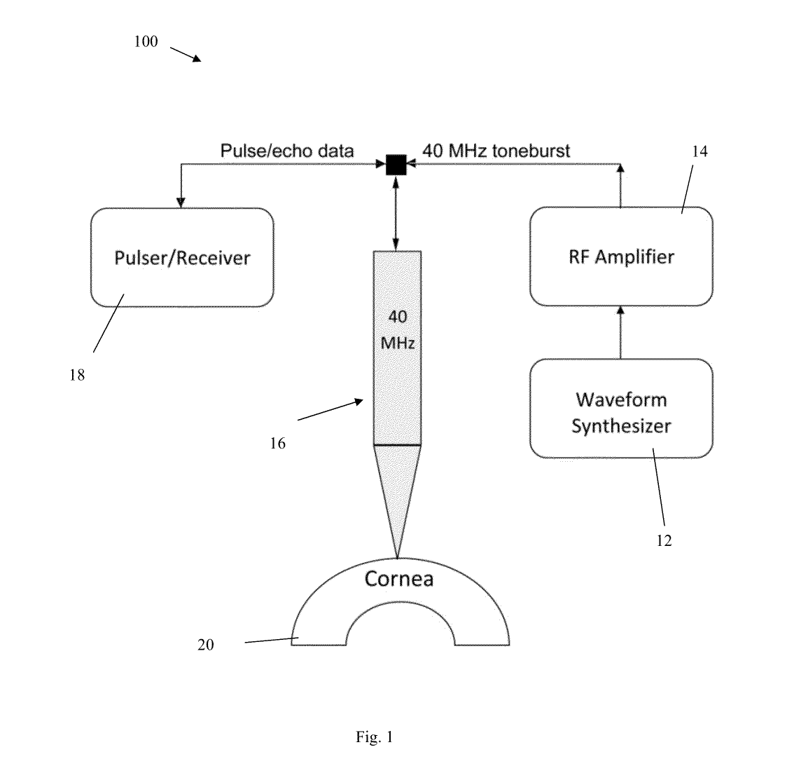

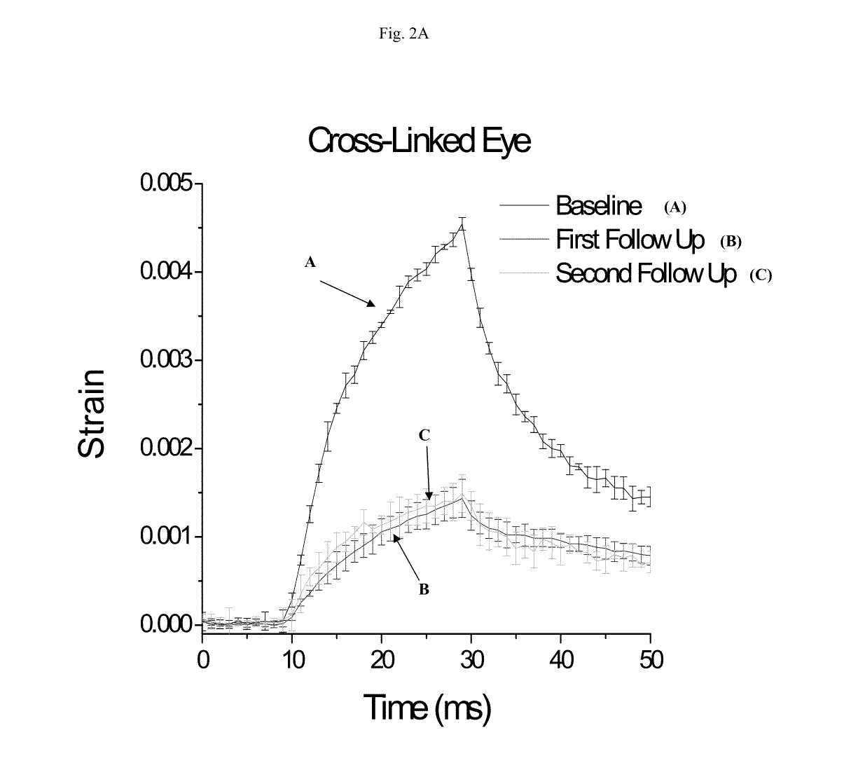

For purpose of explanation and illustration, and not limitation, an exemplary embodiment of the system in accordance with the disclosed subject matter is shown in FIG. 1 and is designated generally by reference character 100. As shown in FIG. 1, system 100 generally includes a waveform synthesizer (e.g., signal generator) 12, an amplifier 14, a transducer 16, and a pulser/receiver 18. The system may be used to provide an acoustic radiation force to a tissue, e.g., the cornea (20). Absorption of ultrasound energy is manifested as a compression force that results in displacement of the cornea as a whole and corneal thinning. The corneal response to the compression force provides information related to its viscoelastic properties.

The ultrasound system includes a focused, transducer (e.g., single element transducer) 12 with center frequency of between approximately 25 megahertz and 50 megahertz, an arbitrary waveform generator (e.g., signal generator) 12, an amplifier 14, a pre-amplifier, and a digitizer. The transducer converts voltage transients into acoustic energy. The arbitrary waveform generator produces waveforms that are amplified (e.g., by a radiofrequency [RF] power amplifier) and then used to excite the transducer. For diagnostic information, the waveform generator produces single sine waves centered at the center frequency of the transducer. After the transducer is excited, echoes are produced where the focused ultrasound wave encounters interfaces between media of differing acoustic impedance (density×speed-of-sound). The pressure waves from echoes are then converted by the transducer into voltages that are linearly amplified by the pre-amp and digitized as RF echo data for subsequent analysis.

To obtain elasticity information, the transducer is first excited periodically (for instance at a rate of 1000/sec) by either a voltage spike or monocycle, and echo data are recorded such that the range to both corneal surfaces and Bowman's membrane are determined. After about 100 such pulse/echo events, the transducer is excited by a toneburst (continuous sine-waves at the center frequency) lasting a fraction of the duration between successive diagnostic pulses, such that sufficient time remains for pulse/echo data to be acquired between successive tonebursts. For example, if 1000 diagnostic pulses are emitted per second, the period between successive pulses is 1000 microseconds. If the focal length is 2 cm, two-way travel time is 2×0.02 m/1540 m/s=26 microseconds. Thus, a minimum of 26 microseconds must be allotted during each 1000 period for a diagnostic pulse, with the remaining time (974 microseconds) available for the toneburst, since otherwise the toneburst would interfere with the diagnostic ranging pulse/echo.

After a series of interleaved diagnostic pulses and tonebursts typically lasting from 1 to 10 milliseconds, the system reverts to diagnostic pulse mode for sufficient time to allow tissue recovery (on order of 100 milliseconds). The initial period of diagnostic pulses allows establishment of baseline conditions, the period of interleaved diagnostic pulses and tonebursts allows examination of displacements during compression, and the subsequent period of diagnostic pulses allows examination of tissue recovery. Recovery of the stromal and epithelial layers of the cornea may occur and different rates, which can be of further use in assessing the individual elastic properties of each tissue separately from the other.

Post-processing of echo data consists of determination of the range to each corneal interface and application of cross-correlation methods between successive echo traces to determine displacements of each interface and change in corneal or corneal layer thickness with time. A spline-fit algorithm is applied to displacement data to obtain sub-micron precision. See generally Francesco Viola & William F. Walker, “A Spline-Based Algorithm for Continuous Time-Delay Estimation Using Sampled Data,” 52 IEEE Transactions on Ultrasonics 80 (2005). Change in thickness of the corneal tissues may be computed by tracking the displacement of the anterior and posterior surfaces of the cornea. Because change in thickness represents strain and the acoustic radiation force absorbed by the cornea is stress, elasticity (stress/strain) is obtained.

In one series of experiments on a rabbit, a 25 megahertz, 0.75 inch focus single-element transducer (Panametrics PZ25-0.40″-SU-R1.50″) was used. For stiffness measurements the following scan pattern was chosen. The transducer was excited at its central frequency by an arbitrary waveform generator (Model WW1281A, Tabor Electronics, Israel) in combination with a broadband RF amplifier (ENI Model 150). The waveform included imaging impulses (25 MHz monocycles) and pushing pulses (25 MHz tone-bursts) at 1 kHz PRF, the sequence of which are (a) 10 imaging impulses (b) 20 pushing pulses applied at 25% duty cycle, with imaging impulses interleaved in the dead time between tone-bursts and (c) 400 imaging impulses following the push mode. This pattern establishes baseline conditions followed by a 5-msec push and 400-msec recovery.

The transducer's acoustic field was characterized by a 40 μm diameter needle hydrophone (Precision Acoustics, UK) calibrated up to 60 MHz. The transducer was excited by a 10 cycle tone-burst. The 6 dB beam width was measured to be 270 μm. The acoustic field parameters of the 25 MHz transducer, i.e., Derated Spatial-Peak Temporal-Average Intensity (I_{SPTA.3}) and Derated Spatial-Peak Pulse-Average Intensity (I_{SPPA.3}), were determined and are tabulated in Table 1 (shown below). These parameters were well within the ranges provided in the relevant FDA 510(K) standards.

TABLE 1

| | Mechanical Index | I_{SPTA.3}(mW/cm²) | I_{SPPA.3} (W/cm²) |
|---|---|---|---|
| Output | 0.05 | 12.6 | 2.53 |
| FDA 510(K) limits | 0.23 | 17 | 28 |
| for ophthalmology | | | |

The intensity was measured as 85.45 W/cm². For stiffness measurements, the tone burst was applied at 25% duty cycle and the temporal average intensity (I) of the beam was 21.36 W/cm².

The acoustic radiation force F [kg/(s² m²) or N/m³] is given by

$$F = \frac{2 α I}{c} \qquad ( 1 )$$

where c [m/s] is the sound speed, α [Np/m] is the absorption coefficient of the tissue, and I [W/m²] is the temporal average intensity at that spatial location. Using a value of 1640 m/s for c and 0.93 db/(cm-MHz) or 267.6 Np/m at 25 MHz for a, F was calculated to be 69741.6 N/m³.

The stress (Pa) on the cornea due to acoustic radiation force is then given by

σ_{A}=F×t (2)

where t is the corneal thickness.

In the presence of boundary conditions, in thin tissues such as the cornea, radiation pressure at the boundary produces a net force

$${F}_{Net} = \frac{{\int }_{A} I d A}{{c}_{1}} [ 1 - \frac{{c}_{1}}{{c}_{2}} + {R}^{2} ( 1 + \frac{{c}_{1}}{{c}_{2}} ) ] \qquad ( 3 )$$

where the subscripts 1 and 2 refer to the two sides of the reflecting boundary, and A is the cross-sectional area of the beam. The reflection coefficient, R, accounts for the multilayer structure of the imaged medium. Assuming 1540 m/s and 1640 m/s for c1 and c2 and 1 gm/cm³ and 1.05 gm/cm³ as the density of saline and cornea, R was calculated to be 0.055. The spot size for a beam width of 270 μm was calculated to be 5.72×10⁻⁸ m².

The net stress (Pa) is then given by:

$${σ}_{Net = \frac{{F}_{Net}}{A}} \qquad ( 4 )$$

which was calculated to be 9.3 Pa. The total stress (Pa) on the cornea would then be given by

σ=σ_{A}+σ_{Net} (5)

Baseline measurements were made on both eyes of the rabbit before treatment. The right eye of the rabbit was then treated with aliphatic β-alcohols, twice a week, for 2 weeks. A follow-up exam was performed after one and two weeks of treatment.

Under anesthesia the eyes of the rabbit were proptosed. Three ARF scans of the cornea, on three different spots were recorded with the 25 MHz ultrasound transducer. A spline based algorithm was used to determine continuous displacement of the front and back surfaces of the cornea to determine the change in corneal thickness. Corneal thickness was measured, assuming a constant corneal speed of sound of 1640 m/s and strain was computed. Strain was plotted as a function of time and fit to the Kelvin-Voigt model:

$$ɛ = \frac{σ}{E} ( 1 - {e}^{- \frac{Ex}{η}} ) \qquad ( 6 )$$

where σ is the total Stress(Pa), E is the Young's Modulus(Pa) and η is the Viscosity (Pa-s).

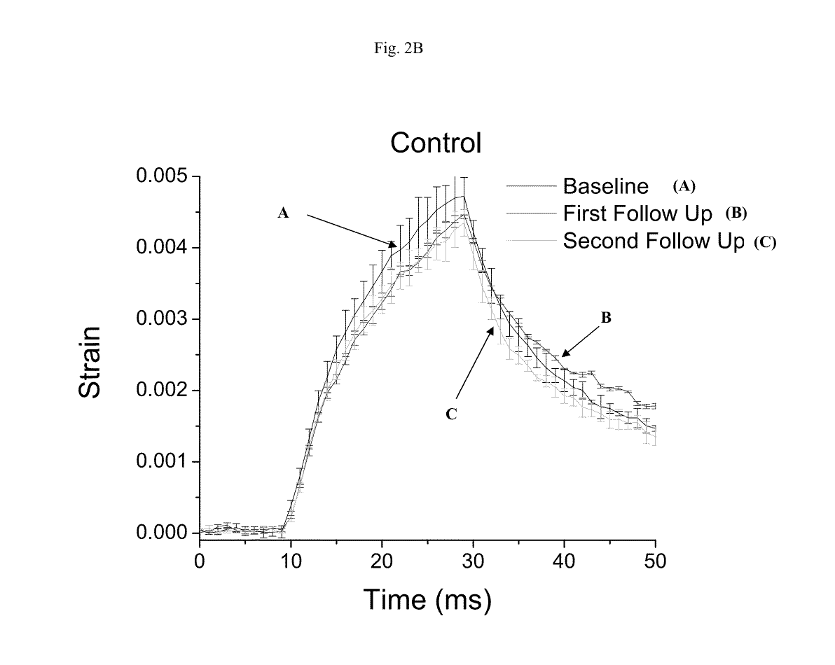

ARF was applied for 5 ms, at 25% duty cycle over a period of 20 ms (from 10 ms to 30 ms on the graph). FIGS. 2A-B, which are plots of acoustic radiation force as a function of time, shows a much lower strain after treatment, indicating that treated eyes were stiffer. These strain values were fit to equation 6. Corneal thickness, stress applied due to ARF, and resulting Young's Modulus values are tabulated in Table 2 and Table 3. Corneal thickness decreases exponentially during the push mode and then relaxes to its original thickness during the next approximately 80 milliseconds. Push data is fit to a second order exponential. Strain and Young's/elastic modulus may be calculated according to the asymptote of this fit. Strain fits to the Kelvin-Voigt model (R2>0.98), yielded a value of 7 kPa for the normal untreated eye. After one week of treatment, the cornea was swollen to three times its original thickness, and the Young's modulus increased to 53 kPa, indicating that the cornea was stiffer. Two weeks post treatment, the cornea was twice the original thickness and the Young's modulus was 35 kPa.

TABLE 2

| | Baseline | | | | | |
|---|---|---|---|---|---|---|
| | | Total | First Follow Up | | Second Follow Up | |
| | Corneal | Stress due | Corneal | Total Stress | Corneal | Total Stress |
| | Thickness | to ARF | Thickness | due to ARF | Thickness | due to ARF |
| Rabbit | (μm) | (Pa) | (μm) | (Pa) | (μm) | (Pa) |
| R750-OD | 385 ± 8 | 36.11 ± 0.10 | 1363 ± 7 | 104.34 ± 2.22 | 608 ± 8 | 51.73 ± 0.08 |
| (cross-linked) | | | | | | |
| R750-OS | 374 ± 9 | 35.36 ± 0.13 | 376 ± 8 | 35.53 ± 0.05 | 386 ± 6 | 36.19 ± 0.13 |

TABLE 3

| | Baseline | First Follow Up | Second Follow Up |
|---|---|---|---|
| Rabbit | Modulus (kPa) | Modulus (kPa) | Modulus (kPa) |
| R750-OD | 7.01 ± 0.43 | 53.10 ± 5.67 | 34.53 ± 3.71 |
| (cross-linked) | | | |
| R750-OS | 7.57 ± 0.38 | 6.74 ± 0.54 | 7.35 ± 0.56 |

In another experiment, a single-element lithium niobate transducer was used that had a center frequency of 35 MHz, 6 mm aperture, and 12.8 mm focal length. Transducer output power was determined by use of a calibrated 40 μm diameter needle hydrophone.

The cornea was examined using an immersion technique with the rabbit under general anesthesia and the central cornea at normal incidence to the ultrasound beam in the focal plane. The ultrasound transducer was excited at its central frequency by an arbitrary waveform generator (Model WW1281A, Tabor Electronics, Israel) in combination with an ENI Model 150 RF amplifier. The waveform included imaging impulses (35 MHz monocycles) and pushing pulses (35 MHz tonebursts) at 5 kHz PRF, the sequence of which was (a) 10 pushing pulses applied at 75% duty cycle, with imaging impulses interleaved in the dead time between tonebursts and (b) 400 imaging impulses following the push mode. This pattern established baseline conditions followed by a 2-msec push and 80-msec recovery.

The digitized RF data were processed with custom software developed in MATLAB (Natick, Mass., USA). The Viola and Walker spline based algorithm was used to estimate the displacements of the anterior and posterior surface of the cornea, between subsequent RF data lines to calculate the change in thickness. This algorithm converts the digitized RF signal to a continuous spline (tracking signal) and uses a pattern matching function to match the subsequent RF signal to the now continuous tracking signal. This allows determination of sub-sample displacements. The difference in displacements of the two surfaces of the cornea is the change in thickness of the cornea. Elasticity was determined using the stress and strain measurements.

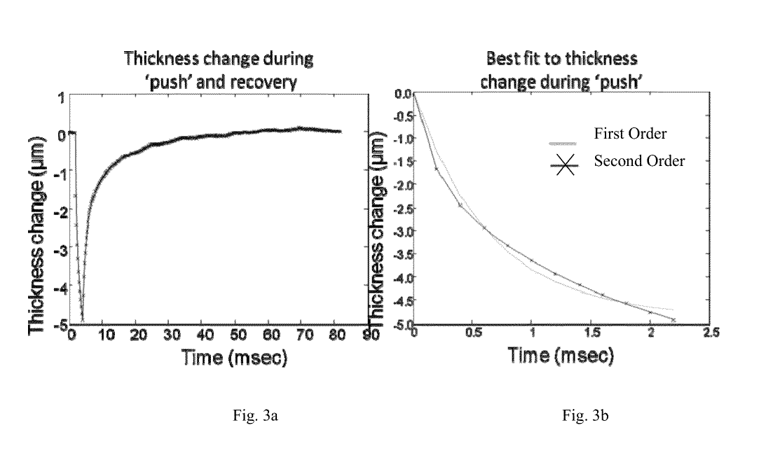

FIG. 3a shows change in corneal thickness after a live rabbit was exposed to the scanning pattern described above. The initial thickness was 366.2 μm and the maximum change in thickness was 4.92 μm, yielding a strain of 0.013. Specifically, thickness decreased during the 2 ms push mode. The cornea relaxed back to its original size during the next 80 milliseconds. The continuous spline based algorithm allows detection of sub-sample size displacement. FIG. 3b is a plot of corneal thickness change during the push, with first and second order exponential best fit to the data. The better fit of the second order equation suggests separate elastic moduli for the epithelium and stroma.

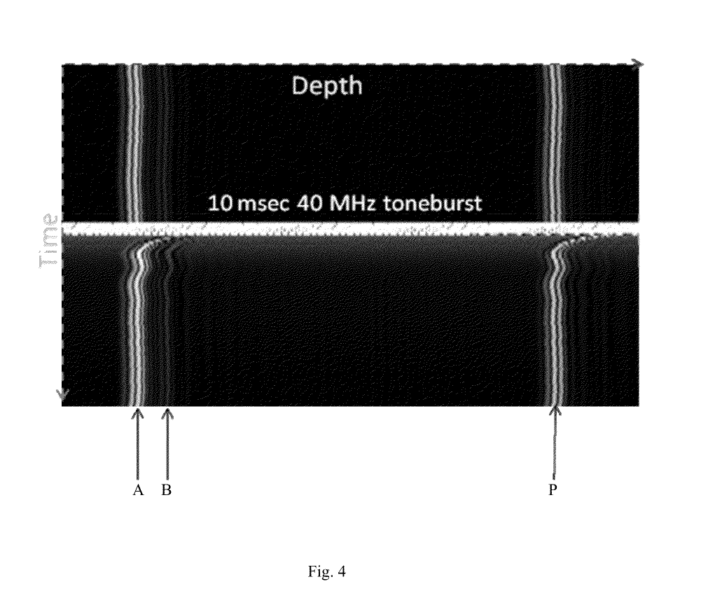

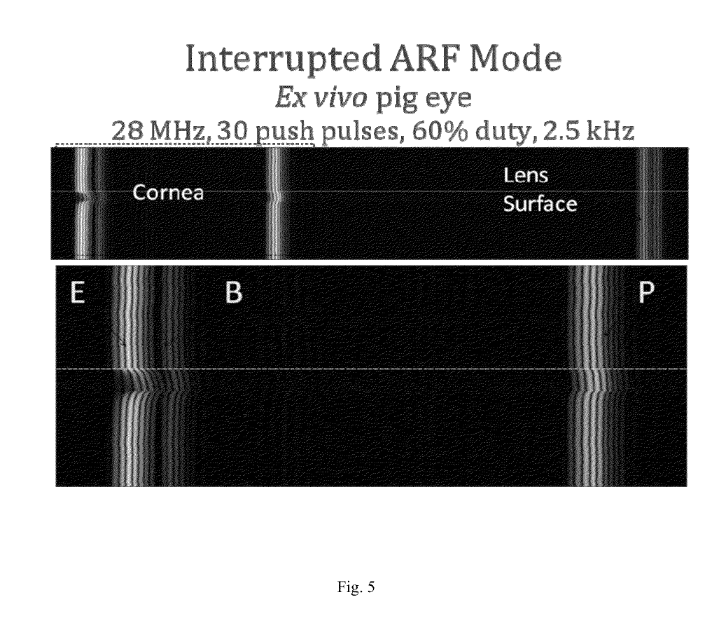

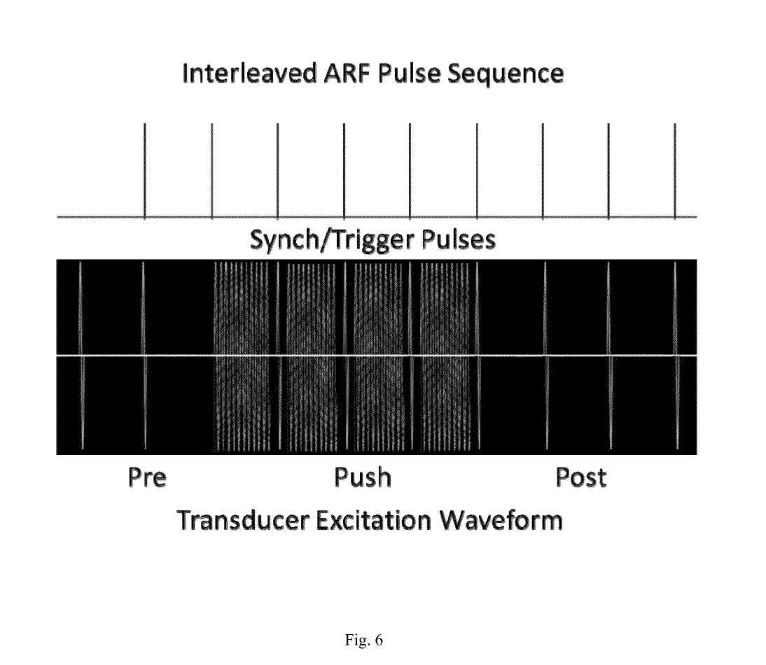

While it is possible to use separate push and diagnostic wavelengths, it may be beneficial to use the same transducer for both the push and pulse/echo. During the push, the corneal tissues are compressed. However, while the push pulse is active, echo data are not obtainable, as illustrated in FIG. 4, and only the recovery can be visualized. Specifically, FIG. 4 is an ultrasound image of one line-of-sight through the cornea as a function of time, with time represented on the y-axis and depth on the x-axis. The continuous 5-msec 40-MHz toneburst produces a white horizontal line as it interferes with acquisition of pulse-echo data. Ultrasound-induced displacements of the anterior corneal surface (A), Bowman's membrane (B), demarcating the epithelial/stromal interface, and the posterior corneal surface (P) are readily apparent. By interleaving push pulses between diagnostic pulse/echo sequences, displacements can be visualized during the push, as illustrated in FIG. 5, which is an ultrasound image of one line-of-sight through the anterior segment of the eye as a function of time, with time represented on the y-axis and depth on the x-axis. The upper image shows the corneal surfaces on the left, beneath which is the fluid-filled anterior chamber of the eye, and then the anterior surface of the crystalline lens. The lower image shows the cornea in greater detail. The 12-msec 28 MHz toneburst used here has the diagnostic pulses and tonebursts interleaved so that we can observe displacements taking place during the course of the ‘push’. Ultrasound-induced displacements of the anterior (epithelial) corneal surface (E), Bowman's membrane (B) and the posterior corneal surface (P) are readily apparent. An example of an interleaved ARF pulse sequence transducer excitation waveform, including synch/trigger pulses and push pulses interleaved with diagnostic pulses is shown in FIG. 6.

In another experiment, Acoustic radiation force (ARF) was directed at the rabbit cornea in vivo, using a single element ceramic (PZT) transducer (28 MHz central frequency, 0.25 in aperture and 0.75 in focal length). Ten pushing pulses were applied at 60% duty cycle, at 2.5 kHz PRF, with imaging impulses and echo return within the interval between push pulses to allow radiofrequency (RF) data acquisition. After the push sequence, the cornea was imaged for another 80 milliseconds. RF data was sampled at 400 MHz (12 bits/sample). A 40 μm diameter needle hydrophone calibrated up to 60 MHz was used to characterize the acoustic field. Allowing for the beam characteristics and the attenuation coefficient of the cornea, the stress applied to the cornea was 80 Pa. Continuous displacement of the front and back surfaces of the cornea was computed with a spline based algorithm to determine the change in corneal thickness. Experiments were performed twice on the same rabbit. Measurements were made on 3 spots on the cornea. Using the techniques described above, it was determined that corneal thickness decreased exponentially during the push mode and then relaxed exponentially to its original thickness during the next 80 ms. Push data was fit to a second order exponential, yielding an asymptote (maximum change in thickness) of 3±0.2 μm. Using the asymptote from the push data, the strain was calculated to be 0.011±0.0008 and the resulting elastic modulus was 10.94±0.85 kPa. Interrupted ARF pulses allowed detection of corneal displacement during force application. Spline based algorithm allowed sub-sample displacement detection of the corneal surfaces, permitting more accurate determination of corneal thickness. Initial studies have shown that the ARF induced change in corneal thickness is dependent on the intraocular pressure.

Devices, systems and methods in accordance with the present subject matter may also be used in other medical applications. For example, the present subject matter may be used to study Age-related Macular Degeneration (AMD). AMD develops in 200,000 people each year in the US and is the principle cause of blindness in those 60 years of age or older in North America and Europe. According to the National Eye Institute, AMD causes visual impairment in an estimated 1.7 million of the 34 million Americans over age 65. As a major public health issue for older Americans, improved early diagnosis and clinical assessment will have a profound impact on patient management and quality of life.

Early AMD is characterized by a spectrum of changes in the ageing eye before overt loss of central vision. These include drusen, focal yellowish-colored extracellular deposits located between the basal lamina of the retinal pigment epithelium (RPE) and the inner collagenous layer of Bruch's membrane, and alterations in macular pigmentation. Late-stage AMD is characterized by neovascularization, causing acute exudative pathology, and/or geographic atrophy (GA), characterized by breakdown of photoreceptor cells and supporting tissue in the central retina. The primary means for diagnostic imaging of the retina are fundus photography, fluorescein angiography (for assessment of the microvasculature), autofluorescence imaging (for assessment of lipofuscin accumulation in RPE) and optical coherence tomography (OCT) (for cross-sectional imaging of the macula, including visualization of choroidal neovascular membranes, layer thinning, and exudative separation of layers). These techniques are useful for diagnosis and assessment of response to therapy, which today mainly consists of intravitreal vascular endothelial growth factor (VEGF) inhibitors such as ranibizumab.

Tissue elastic change may be a precursor to neovascularization. Furthermore, abnormalities in collagen or elastin in Bruch's membrane, the outer retina or the choroid, may predispose towards GA. Microvascular changes, drusen deposits and alterations in elastin may all contribute to elastic changes in the retina/choroid. While a variety of methods now exist for in vivo elastography, these are inapplicable to the retina/choroid either because of insufficient resolution or anatomic inaccessibility to direct compression. The present subject matter includes technologies that non-invasively reveal elastic changes to the retina and choroid. The capability of imaging elastic properties at the level of Bruch's membrane will provide new insights into the pathobiology of AMD and offers a new diagnostic technique for detection of changes associated with disease development, progression and response to therapy.

OCT is currently the primary means for cross-sectional imaging of the retina, providing real-time images with <10 μm resolution. In OCT, a low coherence light source is split into reference and measurement arms of a Michelson interferometer. When recombined, interference between the reference and measurement beams over the laser coherence length produces an A-scan, where range to optical reflectors in the target tissue is provided by varying the range of the reference mirror. In time-domain OCT, range scanning is performed mechanically, limiting acquisition speed. In spectral-domain OCT, the broadband interference is broken into a spectrum using a grating or linear detector array and depth determined from the Fourier transform of the spectrum without movement along the reference arm. The use of a frequency-swept light source for spectral domain OCT imaging of the retina has also been implemented. The combination of an ultra-broadband light source plus frequency domain signal processing has improved resolution to ˜3 μm axially. Scanning speeds of over 300,000 vectors/sec are possible. Also, by acquiring frequency-domain data closer to the eye, an inverted image is obtained in which deeper structures are placed closer to zero delay. This ‘enhanced-depth’ mode allows visualization of the choroid despite absorption by the RPE.

However, neither OCT nor any of the other current ophthalmic imaging modality is directly sensitive to tissue elastic properties. The present subject matter addresses, among other things, this limitation. The subject matter includes a high-speed, high-resolution, phase-resolved OCT to image the retina and choroid as they undergo tissue displacements induced by acoustic radiation force. The subject matter further includes visualizing tissue response using two techniques: acoustic radiation force (ARF) imaging and vibro-acoustic imaging (VAI).

Ultrasonic (US) elastography is typically performed by compressing tissue and observing tissue displacements ultrasonically. If the probe is pressed against the body surface, scatterers along each line of sight can be tracked as the tissue deforms. Deformation is dependent upon the inherent stiffness of each tissue region as well as boundary conditions. Local strain is estimated from the axial gradient of displacement between pre- and post-compression echoes, generally implemented by cross-correlation methods.

Direct compression, however, is impractical for elastography of deeper tissues. In ARF, diagnostic levels of acoustic radiation force are used to remotely induce tissue displacements. The force generated by an ultrasonic beam results from transfer of momentum from the beam to the tissue through absorption and scattering. For a perfect reflector, F=2SE, where F is the radiation force experienced by the tissue, S is reflected power and E is temporal average intensity. In soft tissues, most force is generated via absorption, and for this case, F=AE, where A is the absorbed radiation. Thus, assuming only linear propagation, F=2αIc, where α is the absorption coefficient (m⁻¹), I is temporal average intensity (W/cm²) and c is the speed of sound (m/s). At relatively modest intensities, displacements on the order of tens of microns will occur along the beam axis. For a pulse duration of 28 μsec and 15% duty cycle at 7.2 MHz as the source of the radiation force, with a total duration of 0.7 msec per push and pulse repetition frequency of over 5 kHz. Assuming a typical tissue attenuation coefficient, α of 0.5 dB/cm/MHz and a specific heat, C of 4.2 Jcm⁻³/° C., the temperature rise for this exposure is calculated to be only 0.14° C. using the bioheat equation while neglecting convection and conduction, i.e., ΔT=2αIt/C. ARF has been used to assess hepatic lesions, renal tumors, enhancing contrast in visualization of peripheral nerves among many others.

VAT is another approach using ultrasonic radiation for elastography. In VAT, two transducers with overlapping foci are excited at frequencies ω₁ and ω₁+Δω (where Δω<<ω₁) and interference between the beams in the focal zone generates an oscillating acoustic field (‘beats’) of frequency A w. The low-frequency modulation vibrates the tissue, producing an acoustic field with amplitude proportional to stiffness. In VAI, the beat frequency is typically in the audio range (e.g., 50 kHz) and is detected with a hydrophone. The spatial resolution of VAI is beamwidth laterally and depth-of-field axially. VAI has been used for imaging mass lesions in the breast and liver, for imaging brachytherapy seeds in the prostate and monitoring of cryotherapy.

Acoustic radiation force is used to remotely induce retinal/choroidal compression or vibration. This approach will provide images of a tissue property relevant in the AMD disease process that has heretofore been invisible to all imaging modalities.

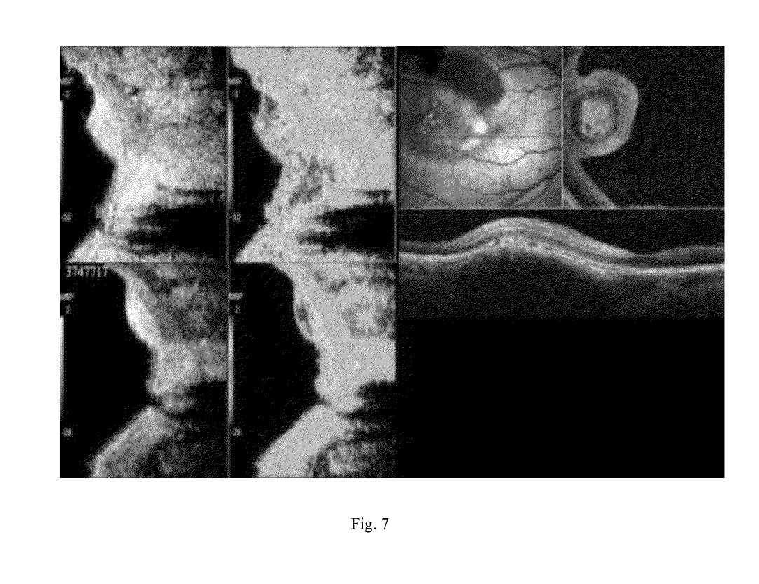

In one evaluation, a focused two-element annular-array transducer with a 36-MHz inner ring and 18-MHz outer ring was used. The outer element was excited with an 18 MHz monocycle and received with the inner (36 MHz) element to improve sensitivity to harmonic signals. The device was characterized and tested using hydrophone data, phantoms, in vivo rabbit eyes, ex vivo pig eyes, and human subjects, demonstrating a 6 dB gain in sensitivity compared to conventional single-element transducers. FIG. 7 shows midband-fit images (6.3 mm depth) of a small choroidal melanoma derived from (top) a single-element 20-MHz transducer and (bottom) a dual element 17-MHz (emit) 36-MHz (receive) transducer. Images on right include scanning laser ophthalmoscope image (grayscale) and OCT C- and B-modes of tumor. OCT images do not penetrate beyond retina overlying the tumor. The dual element harmonic image shows improved resolution and depiction of tissue layers compared to fundamental 18-MHz image obtained with single-element probe.

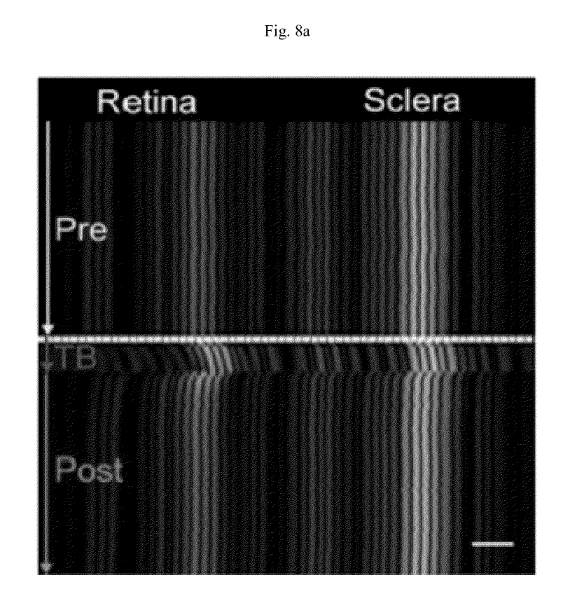

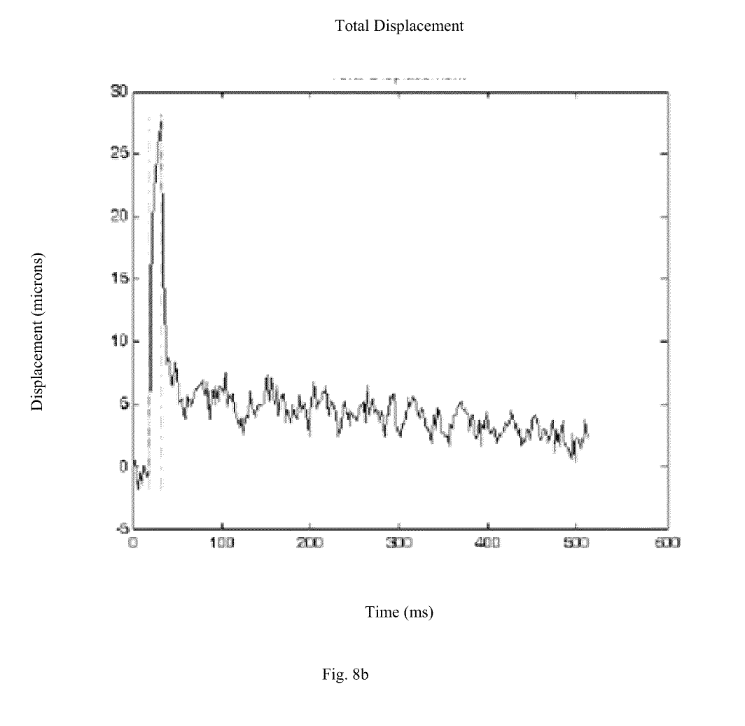

Using a dual-element transducer of the kind described above for retinal imaging, experiments were conducted to verify that tissue displacements could be produced in ocular tissues using ultrasound (US) force levels at intensities within FDA guidelines for the eye. After establishing baseline conditions for 100 consecutive pulses along one line-of-sight, we interleaved force-generating 18-MHz tone bursts 1.8 msec in duration (90% duty cycle) between successive monocycles over a total period of 12 msec (6 cycles). The interleaving process allowed sufficient time between successive tone bursts to obtain 36 MHz pulse/echo data so that displacements could be measured during the course of exposure. The system then reverted to monocycle excitation to record the recovery. FIG. 8a shows an M-scan demonstrating tissue displacements at the posterior pole of an ex vivo rabbit eye. Time is in the vertical axis, proceeding from top to bottom, and range is in the horizontal axis. The scale bar represents 100 μm. The toneburst (TB) begins simultaneously with a diagnostic acoustic pulse, causing the horizontal white line marking initiation of tissue compression. Thereafter, tonebursts of 1.8 msec are interleaved between successive diagnostic pulses that are 2 msec apart, allowing displacements to be seen without interference from the tonebursts. FIG. 8b illustrates displacements measured at the retinal pigment epithelium show a sharp immediate compression followed by an initial rapid ˜80% recovery and a long gradual return to baseline over >0.5 sec.

Echo data were digitized at 400 MS/s (12-bit resolution). A spline-based algorithm was used to process data to determine the magnitude and time course of displacements in retina in a fresh ex vivo rabbit eye. Maximum displacements measured 12.4 and 25.8 μm at ultrasound intensities of 60 and 100 Wcm⁻² (not derated), respectively. Of special note is the increase in amplitude of RPE echo during the toneburst, which is likely a result of increased relative acoustic impedance with compression. This phenomenon may also occur with OCT-monitored compression as a result of refractive index alteration with compression. OCT would also provide much finer anatomic detail given its order-magnitude higher resolution and higher contrast. Finally, the results shown here are for non-perfused tissue. Blood flow would certainly affect in vivo elastic properties.

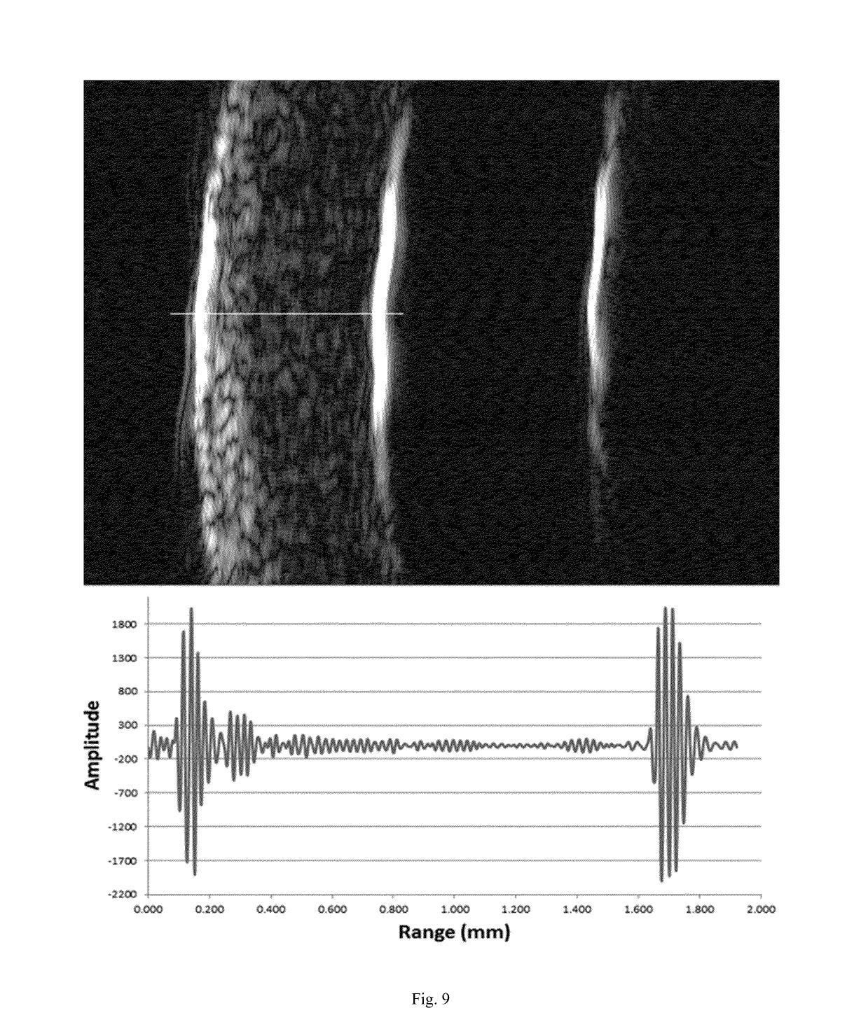

The dual frequency/dual element probe was also used to image the anterior segment of an ex vivo pig eye, as shown in the B-Scan FIG. 9. Harmonic images were obtained by exciting the outer 18 MHz element and receiving with the central 36 MHz element. Harmonic images of the cornea demonstrate enhanced lateral resolution, due to suppression of sidelobes in the harmonic. Furthermore, harmonic images demonstrate enhanced corneal stromal backscatter. This characteristic would enable measurement of intra-stromal displacements when the cornea is exposed to ARF tonebursts from the outer element that were interleaved with diagnostic pulses. This would allow characterization of strain as a function of depth within the stroma.

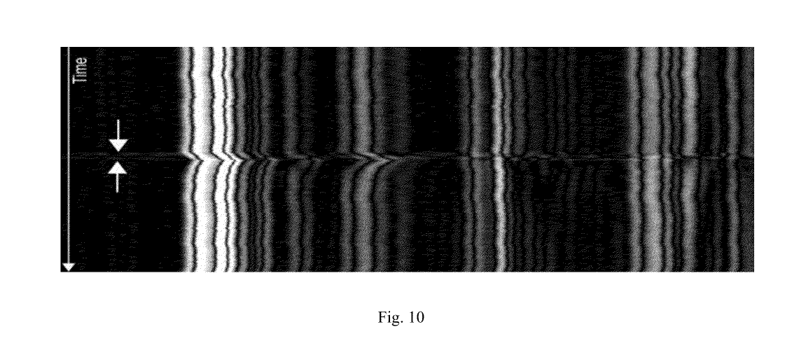

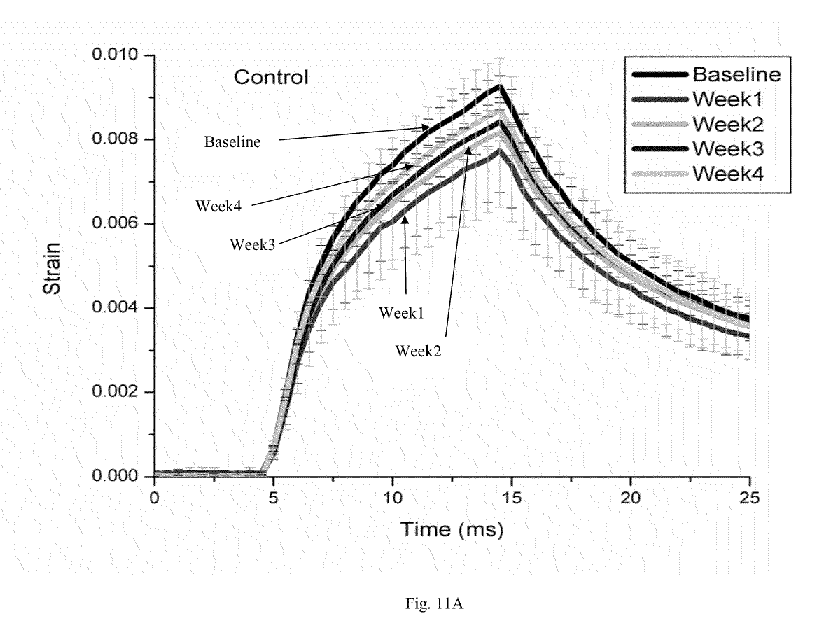

A VAI system was tested using an annular dual-element device of center frequency 865 kHz. The inner and outer rings were excited using two ENI power amplifiers and two Agilent waveform generators. Using excitation tonebursts of 100 pee, both elements were excited at diagnostic power levels, and recorded displacements generated in an agar phantom using a 35 MHz transducer operating in pulse/echo mode (rep. rate=2 kHz). The effect of the beam on the phantom is illustrated in FIG. 10, which is M-mode data acquired at a PRF of 2 kHz showing displacements induced in an agar phantom when exposed to interfering 865 kHz and 866 kHz ultrasound beams. Conventional pulse/echo ultrasound has insufficient repetition rate to adequately monitor VAI vibrations induced at the beat frequency. While the arrangement is not ideal for measuring tissue vibration, it approximates how VAI could be implemented.

Displacements are determined by cross-correlating pre- and post-compression data. Local strain, e, can be calculated from displacements based on some simplifying assumptions: isotropic incompressible medium and uniform axially applied stress. Local strain is then c=(d2−d1)/Δz, where d1 is the displacement at distance z from the top of the target, and d2 is displacement at z+Δz. The elastic modulus is then [symbol] /e, where [symbol] represents the axial radiation force in N/m².

An advantage of the present subject matter is the use of local excitation via focused US. By localizing the excitation, the local strain is to a large extent decoupled from boundary conditions imposed by surrounding tissue and surface topography. This decoupling allows strain to be calculated directly from experimental data, without the implementation of complex models. This permits the focus of algorithm development and signal processing to be on optimally extracting the displacement. Such localized excitation has the potential to impact on other applications of elastography.

For vitroelasticity determination, the two elements of the 10 MHz annular transducer are exited at 10 MHz and 10 MHz+dF, where dF≦50 kHz (which is well within the bandwidth of the probe). Toneburst durations are such that at least 5 cycles at the beat frequency, dF, are encompassed. For instance, if dF=5 kHz, the pulse duration is 1 msec.

Detection of vibroacoustic displacements is facilitated by use of a diagnostic ultrasound wavelength sufficiently separated from vibroacoustic fundamental wavelength so as to avoid interference. Also, temporally-averaged and derated pulse intensity are limited to comply with FDA 510k standards.

Subjects may be scanned using a standard immersion procedure with topical anesthesia. Subjects are placed on an examination table facing upwards. A 3-M 1020 steri-drape, which has a central aperture, is attached to the skin surrounding the eye to form a watertight seal. After instillation of a few eye drops of 0.5% Proparacaine-HCl topical anesthetic, a Barraquer eyelid speculum is inserted to prevent blinking. The steridrape is secured to a ring stand, and warm sterile normal saline solution will be added until the eye is submerged to a depth of ˜2-cm. The US scan assembly is then lowered into the waterbath and scanning is performed with reduced room illumination.

ARF data may be acquired along a single line of sight and observe displacements occurring during and after application of acoustic radiation force. Images are generated representing local displacement. With VAI, vibration amplitude as a function of tissue depth relative to the retinal surface may be determined, thus compensating for the ARF-like component of the acoustic field.

The aforementioned techniques may be useful to study drusen in Bruch's membrane and geographic atrophy. Localized changes of the retina and choroid may be a precursor to continuing extension of geographic atrophy. Identification of a specific “signature” for predisposition to GA would improve understanding of the natural history of atrophic progression. Neovascularization may also be assessed in that reorganization of the choroidal microvasculature results in altered elastic properties. In addition, altered elasticity may act as a precursor to development of neovascularization.

Still other applications of the present subject matter may include assessing elastic properties of other tissues in vivo. For example, the elastic properties of intraluminal tissues may be assessed by placing a device in accordance with the present subject matter on an intraluminal probe. Such an application would be useful for, e.g., determining a degree of plaque within a vessel and how the plaque affects the vessel's elastic properties. Additionally, in the cosmetic, dermatologic, and cosmetic surgery fields, the present subject matter may be employed to assess elastic properties of skin such as the dermis and epidermis.

In accordance with another aspect of the presently disclosed subject matter, an in vivo technique is provided to determine difference in biomechanical strength of the cornea after a collagen cross-linking therapy (CXL). In one experiment, a CXL procedure was performed on the right eyes of six rabbits while the left eyes were used as controls. ARF was used to assess corneal stiffness in vivo, once before treatment (to establish a baseline) and on a weekly basis (for a period of four weeks) after treatment. The cornea was exposed to ARF using a single element transducer having a 25 MHz central frequency; 6 mm aperture; and 18 mm focal length (Panametrics V324-SU). The beam sequence consisted of 20 pushing tonebursts of 400 μs duration (80% duty cycle). Imaging impulses were interleaved in the dead time to allow the same transducer to acquire radiofrequency data during the push mode to image corneal displacement. The acoustic power levels exhibited were within FDA-specified levels for ophthalmic safety. Displacement of the front and back surfaces of the cornea were used to determine the change in corneal thickness and strain. ARF induced strain was fit to the Kelvin-Voigt model to determine the elastic modulus. The average moduli were calculated for the six rabbits, for each of the five time points (i.e. baseline and weeks 1-4).

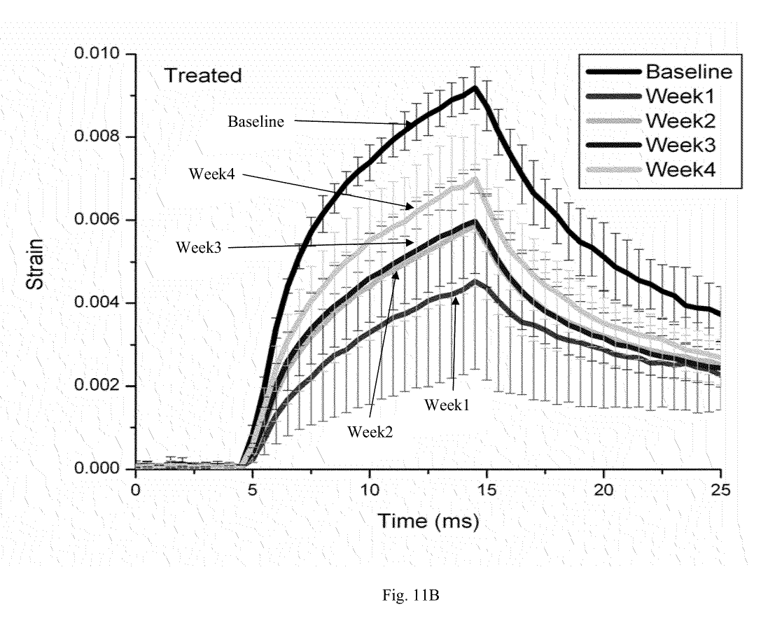

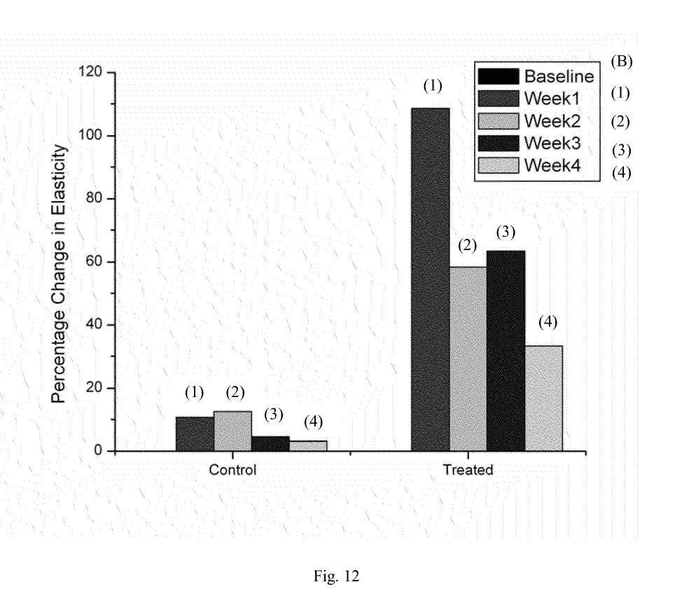

At the end of four weeks, ARF measurements showed an increase of average elastic modulus by 33% in the treated eye, and 3% in the control eye. Paired t-tests revealed statistically significant differences between treated and untreated eyes from week 1 to week 4 (p=0.0005, 0.04, 0.0007, 0.006). There was no significant difference between right and left eyes before treatment (p=0.95). The results of this experiment are graphically illustrated in FIGS. 11A-B, which plots average corneal strain as a function of time during and after exposure to ultrasound radiation force in the control (FIG. 11A) and cross-linked (FIG. 11B) rabbit corneas. Also, FIG. 12 shows the average change in elasticity measured for four weeks in the control and the cross-linked rabbit corneas.

These findings demonstrate statistically significant differences in stiffness between control and CXL-treated rabbit corneas in vivo based on axial stress/strain measurements obtained using ARF. Accordingly, the capacity to non-invasively monitor corneal stiffness, as provided by the system and techniques described herein, offers the potential for clinical monitoring of CXL.

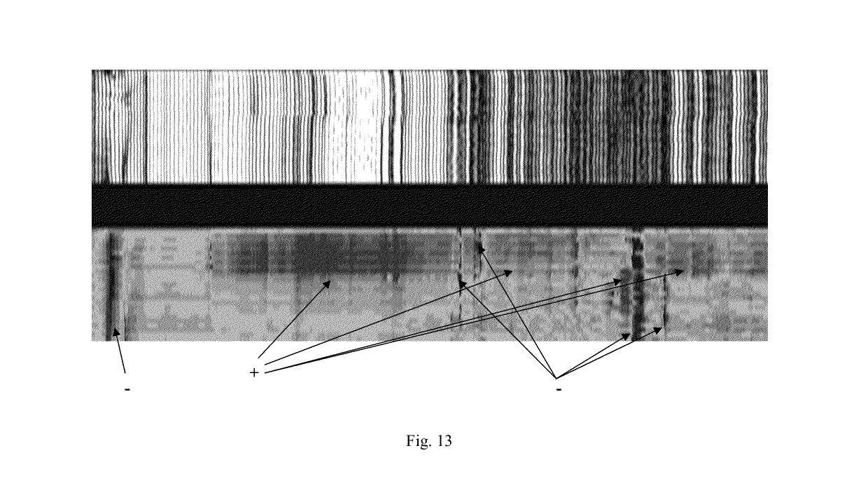

In another study, an 18 MHz single-element transducer was employed having a 31 mm focal length. The eyes were oriented to allow the ultrasound beam to enter the eye anterior to the equator so as to avoid absorption and refraction by the lens. After focusing on the retina, the transducer emitted a series of ten 18 MHz tonebursts at 1 msec intervals with a 25% duty cycle. Radiofrequency pulse/echo data were digitized at a pulse repetition rate of 1 kHz before, during and after ARF. Echo data (32 μm long kernel) during and for 15 msec post-push were cross-correlated with pre-push data over an 80 μm long window to determine ARF-induced displacements during the push and during relaxation. Displacement values were used to generate color-coded displacement images superimposable upon conventional grey-scale images representing echo amplitude. FIG. 13 shows color-coded displacements superimposed upon the B-mode image. Positive displacements are coded red (and additionally denoted by “+” for ease of identification) and negative blue (and additionally denoted by “−” for ease of identification). The red/blue (or +/−) later at the left of the image is actually the choroid, where displacements are actually due to perfusion rather than ARF-exposure. Scleral displacement was minimal, but large strains of up to 16 μm were seen in extraocular muscle and orbital fat, with rapid recovery, usually in 1 or 2 msec, with overshoot to negative displacement common.

Accordingly, the ARF technique disclosed herein provides a non-invasive procedure for assessment of relative tissue stiffness that can be applied to the posterior coats as well as orbital tissues. Using power levels within FDA safety guidelines, this technique offers a means to probe changes in tissue stiffness in the posterior coats and orbital tissues in vivo.

In accordance with another aspect of the presently disclosed subject matter, a study utilizing ARFI (Acoustic Radiation Force Impulse) response to probe functional properties of tissue was conducted using a ultrasound transducer with a center frequency of 18 MHz, a 10-mm aperture, and a 30-mm focal length. Transducer output was characterized using a using a certified 40-μm needle hydrophone calibrated up to 60 MHz (Precision Acoustics, Ltd., Higher Bockhampton, Dorchester, UK).

In the first set of experiments, the rabbit eye was gently proptosed and placed through a hole in a latex membrane, forming a watertight seal and exposing the globe. The membrane was secured to a ring stand to allow formation of a normal saline water bath to provide an acoustic coupling medium between the ultrasound transducer and the eye. A second set of experiments was subsequently performed in which, after completing examination of the proptosed eye, the globe was reseated normally in the orbit and a 6/0 silk suture was attached to the temporal sclera near the equator. The eyelids were then held open with a lid speculum and hypromellose lubricant (GenTeal; Novartis Pharmaceuticals Corp., East Hanover, N.J.) was applied to the surface of the eye. The thread was pulled to rotate the globe so as to expose the equator, and lowered a water-filled polyethylene membrane onto the globe to provide an acoustic coupling medium.

A total of fifteen experimental procedures were performed on six eyes of three rabbits. The effect of proptosis on intraocular pressure (IOP) was determined in a separate series of twelve rabbit eyes. IOP measurements were made using a veterinary tonometer (Tono-Pen Avia Vet; Reichert Technologies, Depew, N.Y.) during and after proptosis. The transducer was acoustically coupled to the eye by submerging its surface in the water bath. The beam axis was oriented nearly normal to the globe between the limbus and the equator, crossing the eye and achieving focus on the posterior layers on the opposite side of the globe. This nonaxial arrangement was utilized to avoid attenuation and defocusing of the ultrasound beam by the lens, which is very large in the rabbit (mean axial dimension of 7.9 mm versus about 4 mm in humans) and possesses a high acoustic absorption coefficient to average about 1.5 dB cm⁻¹ MHz⁻¹, about triple that of typical soft tissues

The ultrasound excitation system consisted of a programmable arbitrary waveform generator (Model WW1281A; Tabor Electronics, Tel Hanan, Israel) whose output (set at 400 mV peak to peak) was amplified by 55 dB by a broadband radiofrequency (RF) amplifier (Model A150; Electronic Navigation Industries, Rochester, N.Y.). The RF signal was passed through a diode expander circuit and then to the transducer to excite ultrasound emission. RF echo data returned through the expander and a limiter (which constitute a protection circuit, shielding sensitive downstream components from the high voltage excitation waveforms) and were then passed to a preamplifier (Model AU1480; MITEQ, Inc., Hauppauge, N.Y.) and to a digitizer (Acqiris model DP310; Agilent Technologies, Monroe, N.Y.). RF echo data were acquired at a sample rate of 400 MHz at 12-bit precision.

The transducer was excited by a series of 18-MHz monocycles (duration=56 ns) at a pulse repetition frequency (PRF) of 1 kHz to establish preexposure, baseline conditions and RF echo data digitized. After 100 such “tracking” pulses, the transducer was excited by a 250-μs long, 18-MHz tone burst (4500 contiguous cycles), which constitutes an ARFI push pulse. Following this, nine additional 250-μs tone bursts were interleaved between monocycles at a 1 kHz PRF (1-ms period) over a total period of 10 ms. This interleaving of tone bursts between tracking pulses allowed acquisition of pulse/echo data during the dead time between tone bursts. (Had ARFI been performed without such interruption, echoes produced by tone bursts would have interfered with acquisition of pulse/echo data.) After the above interleaved ARFI/monocycle excitation, the system reverted to monocycle excitation at a 1 kHz PRF to track recovery.

Data were analyzed by measurement of shifts in acoustic phase fronts in phase-resolved M-scans, which capture echo data along one line of sight as a function of time. In the disclosed system, phase shifts could be measured to minimal value of 2 μm, the spatial equivalent of each digitized RF echo data sample, although upsampling (digital interpolation) can provide subsample precision. Achievable precision, however, is affected by factors such as electronic noise, jitter, and phase decorrelation resulting from physical processes such as respiration and the cardiac cycle. The maximum phase shift determinable without aliasing is a half-wavelength, in this case approximately 42 μm. ARFI-induced phase shifts depict axial tissue displacements in response to compression by radiation force. Phase shifts may also occur due to particle motion (i.e., blood flow). In this case, the shift occurring over a known time interval allows computation of axial particle velocity.

As a result of these experiments, Hydrophone measurements in the focal plane of the ultrasound beam showed a −12-dB beam width of 350 lm. Peak negative pressure was 3.2 megapascals. Derated spatial peak pulse average intensity measured 6.0 W/cm⁻², with mechanical index (MI) determined to be 0.102. For the case of ten 250-μs long ARFI bursts over a period of one second, derated spatial peak temporal average intensity measured 16.3 mW/cm⁻². Accordingly, these values fall within FDA 510(k) standards for ophthalmic diagnostic ultrasound. Under these conditions, the biohcat equation indicates an expected local temperature rise of under 0.48 C, assuming no blood flow and a typical tissue attenuation coefficient of 0.5 dB/cm⁻¹/MHz⁻¹ (Perfusion would further reduce the ARFI-induced temperature increase.) Axial resolution (inverse 12-dB bandwidth) provided by the transducer was approximately 130 μm. The focal depth-of-field was calculated to be approximately 5 mm.

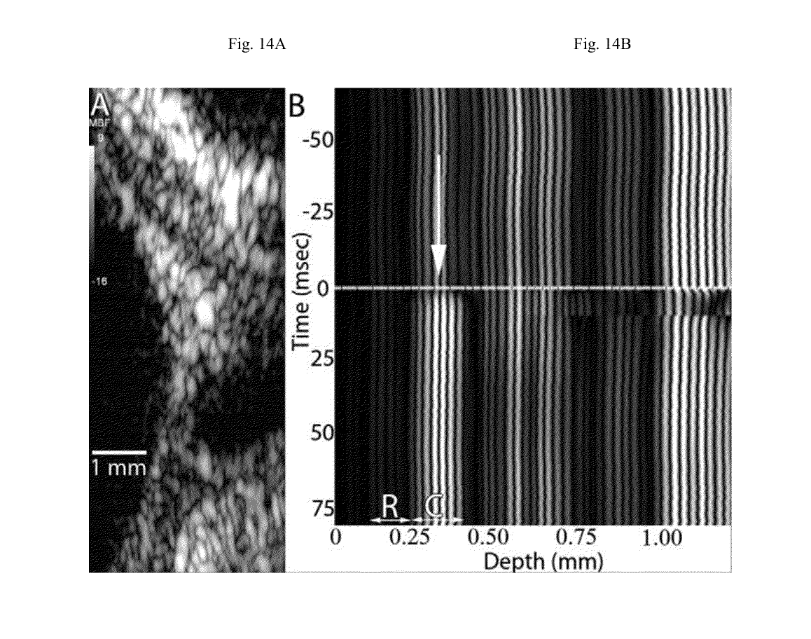

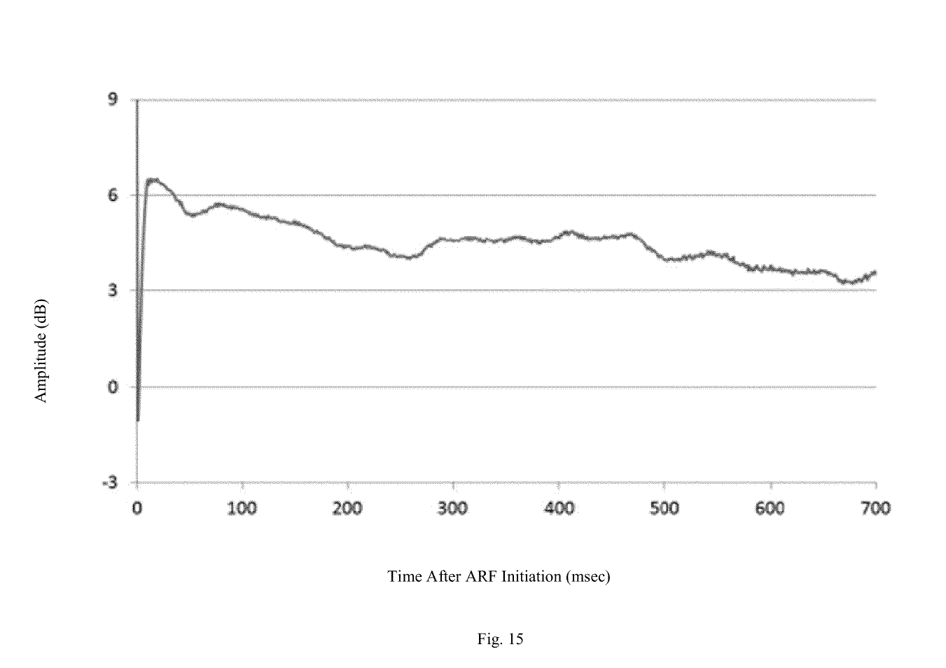

Comparison of IOP before and after proptosis in 12 eyes showed a mean IOP of 11.1±2.1 mm Hg in normally seated eyes versus 30.9±6.5 mm Hg following proptosis. FIG. 14A depicts a B-scan image and FIG. 14B depicts an M-scan that captures the response of the posterior tissue layers to ARFI exposure in a proptosed eye. The y-axis of the M-scan in FIG. 14B represents time and the x-axis represents tissue depth, with each horizontal image line showing echo data along a single line of sight at 1-ms intervals. Additionally, the detail of phase-resolved M-scan in FIG. 14B, demonstrates echo amplitude as a function of depth along a single line of sight over time. The retina, R, is generally faintly reflective compared with the choroid, C. The M-scan shows the response to a 10-ms ARFI exposure, whose initiation is indicated by horizontal line at time=0. The ARFI response consisted of a significant immediate increase in echo amplitude from the choroid (indicated by the large arrow). Qualitatively, an immediate increase in backscatter following ARFI within an approximately 150-μm thick layer that has low reflectivity under pre-ARFI baseline conditions is depicted. This layer lies beneath a faintly reflective superficial layer measuring approximately 125 μm in thickness. This can be interpreted as representing the retina and choroid. A plot of choroidal backscatter amplitude as a function of time is presented in FIG. 15.

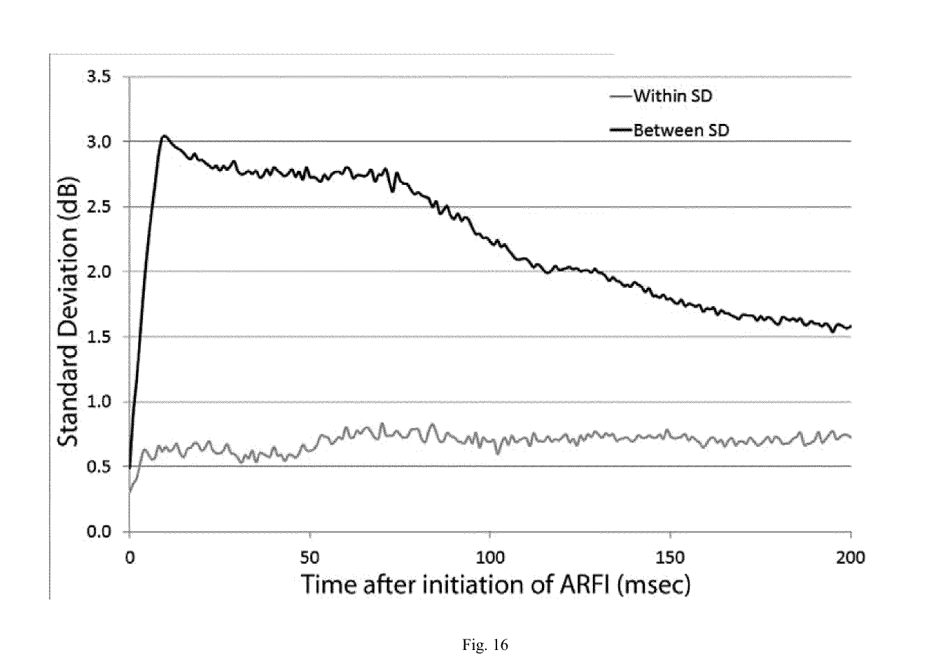

To investigate how this effect varied as a function of position, the eye was subjected to three consecutive exposures at 15-second intervals in a series of 10 positions linearly spaced at 0.1-mm intervals. Mean echo amplitude was measured within the choroid relative to pre-exposure levels as a function of time following ARFI. The standard deviation of echo amplitude as a function of time between and within positions is plotted in FIG. 16. The results demonstrate very high reproducibility of the effect from multiple exposures at one spot, but considerable variation from position to position, with some spots showing a negligible increase in choroidal backscatter and others exceeding 6 dB.

Additionally, the effect of reducing the intensity of the ARFI beam was examined by monitoring changes in choroidal backscatter at one site as the excitation voltage was reduced in a series of 3-dB steps. The results, summarized in Table 4 below, show only a small reduction in peak choroidal backscatter amplitude increase with 3 to 6 dB of attenuation, but a more pronounced drop with increasing attenuation. Similarly, the duration of the ARFI-induced backscatter increase did not decrease until at least 9 dB of attenuation was added.

TABLE 4

| Attenuation | Peak Negative | Peak Backscatter | Decay Time |
|---|---|---|---|
| (db) | Pressure (MPa) | Increase (dB) | (ms) |
| 0 | 3.19 | 8.2 ± 0.3 | 293 ± 64 |
| 3 | 2.83 | 7.8 ± 1.3 | 301 ± 98 |
| 6 | 2.44 | 6.2 ± 1.0 | 315 ± 64 |
| 9 | 2.01 | 3.3 ± 0.6 | 140 ± 50 |
| 12 | 1.60 | 2.2 ± 0.3 | 33 ± 0 |

For further analysis, the duration of ARFI was doubled and halved. However, this demonstrated little effect on the change in backscatter amplitude, but did affect the duration of backscatter, with a 5-ms exposure producing only half the duration of choroidal backscatter increase as a 20-ms exposure.

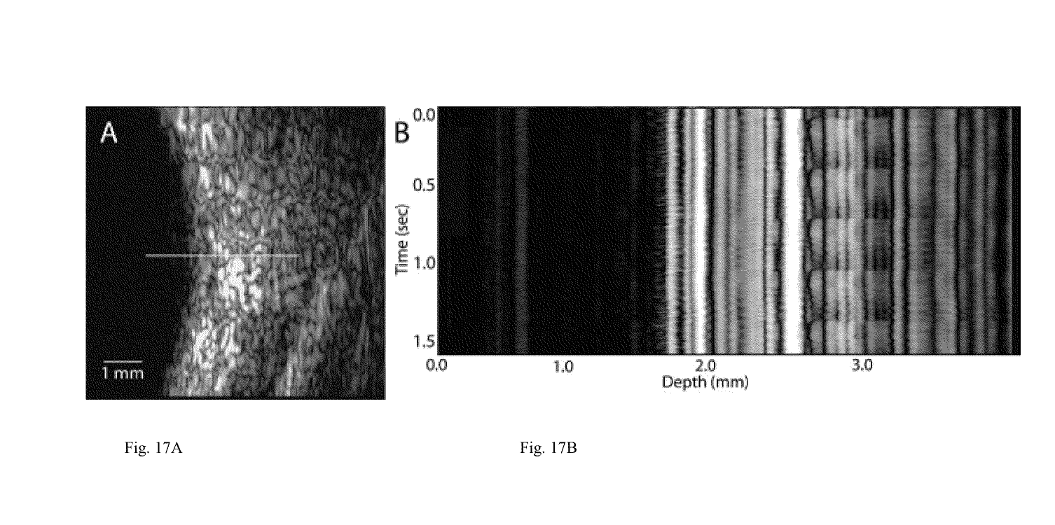

FIG. 17A-B shows B- and M-scans, respectively, of the posterior of the normally seated rabbit eye, demonstrating pulsatile flow in orbital vessels. The M-scan of FIG. 17B corresponds to the horizontal line in the B-scan and demonstrates pulsatile flow in orbital vessels, indicated by periodic backscatter amplitude increases. The pulse rate is approximately 200 cycles per minute.

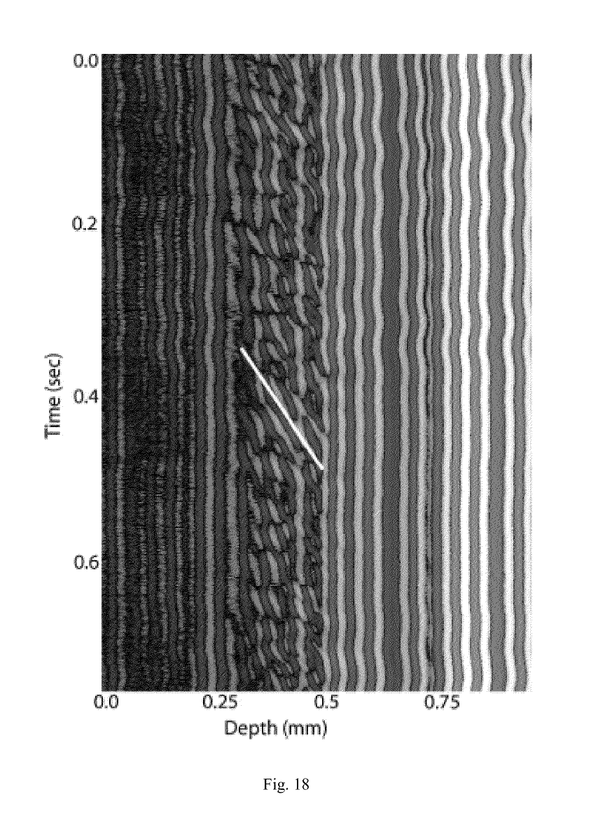

FIG. 18 illustrates perfusion of the choroid. The choroid, which in this case measures approximately 0.2 mm in thickness, shows diagonal phase (see line) contours resulting from particle motion along the beam axis. From the slope of the phase lines, a flow velocity of approximately 1.3 mm/s can be determined. This is an axial velocity, not taking into account the unknown angle of the choroid to the beam axis. Pulsatile choroidal flow is not observed.

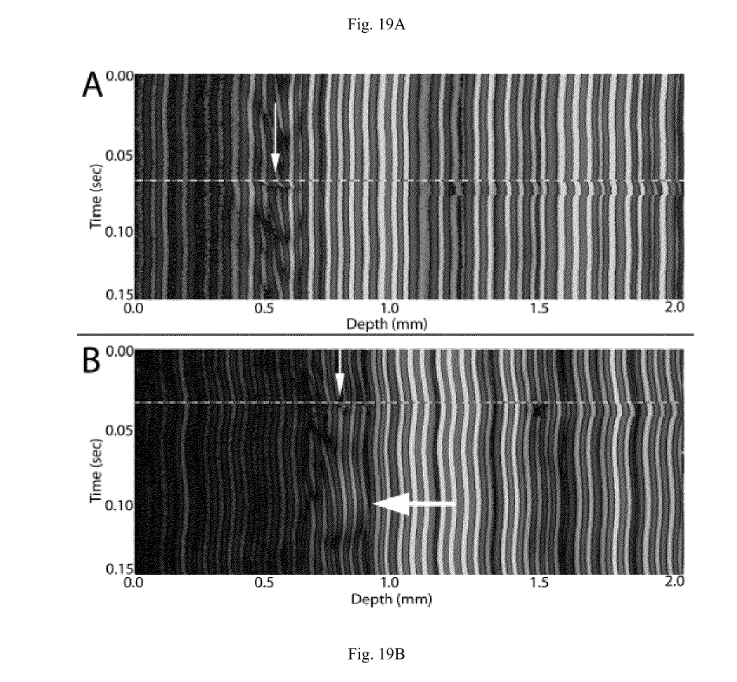

FIGS. 19A-B show the effect of ARFI exposure on two regions of the choroid before, during, and after ARFI. ARFI initiation is indicated by the horizontal broken-line through the figures. Flow within the choroid (small arrows) is detectable as phase shifts that are caused by blood cell particle motion along the beam axis. As shown in FIG. 19A, during the 10-ms ARFI exposure, there is rapid motion of blood cells within the choroid. A typical response to ARFI: unlike the proptosed eye, no post-exposure increase in backscatter is observed. As shown in FIG. 19B, a small increase in backscatter occurs from about 50 to 100 ms after ARFI exposure, but this effect was rarely observed. Accordingly, ARFI exposure typically generated tissue displacements on the order of 10 μm at the margins of the choroid, with larger displacements within the choroid. Displacements in the orbit tended to be larger, about 15 μm, indicative of less stiffness compared with the choroid. The increase in choroidal backscatter observed in response to ARFI in the proptosed eye was either absent or small (as shown in FIG. 19B).

Accordingly, the presently disclosed subject matter employs ARFI to detect the dynamic effects of transient compression and is not limited to the effects induced by static loading. Additionally, the disclosed subject matter describes technique for remote and focused compression of the posterior coats and their elastic and vascular response on a millisecond time scale. Furthermore, ARFI's ability to noninvasively probe axially resolved tissue elastic properties at discrete locations, as disclosed in the techniques described herein, is unique.

Additionally, the disclosed subject matter can be employed to examine the effect of radiation force on the choroidal circulation under conditions of ischemia induced by elevated intraocular pressure.

Indeed, the disclosed subject matter can serve to open many new possibilities for investigation of the elastic and vascular properties of the retina and choroid. For instance, the inner limiting membrane of the retina mediates forces between the retina and vitreous body, and thus its elastic properties may play a role in the pathogenesis of various retinal disorders especially during posterior vitreous detachment. Additionally, the techniques and findings of the disclosed subject matter can be useful in probing the elastic properties of the lamina cribrosa, which plays a central role in glaucoma pathogenesis.

Moreover, while some of the exemplary studies described herein monitored displacements with the same transducer used to generate radiation force, the combination of acoustic radiation force with OCT may be advantageous in that the far higher resolution of OCT would allow visualization of ultrasound-induced displacements within the retinal and choroidal structural layers. Also, the anatomy of the human eye is more amenable than that of the rabbit for ultrasound exposure of the posterior coats due to its much smaller lens and better exposure of the globe. This, plus the safe, diagnostic levels of ultrasound used in the experiments described herein, opens up the possibility of clinical application in pathologies such as maculopathies, diabetic retinopathy, glaucoma, and even myopia.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. Thus, it is intended that the disclosed subject matter include modifications and variations that are within the scope of the appended claims and their equivalents.

1. A medical device for measuring properties of superficial tissue, comprising:
an ultrasonic transducer having a center frequency of at least approximately 25 megahertz;
a signal generator for producing waveforms; and
an amplifier,
wherein the signal generator, amplifier, and transducer are in electronic communication with each other, and
further wherein the transducer is configured to direct acoustic energy toward the tissue in response to the waveforms.

2. The medical device of claim 1 wherein the center frequency is between about 25 megahertz to about 50 megahertz.

3. The medical device of claim 1 wherein the transducer is a single element transducer.

4. The medical device of claim 1, wherein the signal generator is an arbitrary waveform generator.

5. The medical device of claim 4, wherein the waveform generator is capable of interleaving diagnostic pulses with push pulses.

6. The medical device of claim 5 wherein the diagnostic pulses comprise a voltage spike or monocycle.

7. The medical device of claim 6 wherein the push pulses have a frequency approximately equal to the center frequency.

8. The medical device of claim 1, wherein the medical device is capable of determining whether the superficial tissue is semi-superficial tissue.

9. The medical device of claim 8, wherein the superficial or the semi-superficial tissue includes cornea, retina, or skin.

10. The medical device of claim 1, wherein the properties include at least one of viscosity, elasticity, and cross-linking.

11. The medical device of claim 1, wherein elasticity of first and second tissues can be determined.

12. The medical device of claim 11, wherein the first tissue is the epithelium and the second tissue is the stroma.

13. A medical device for determining properties of first and second tissues of the eye, the medical device comprising:
a transducer, the transducer being configured to emit output acoustic waves in response to an input waveform, to create an output waveform in response to input acoustic waves, and to emit pulses at a sufficiently high frequency such that a first echo created at a first tissue in response to the output acoustic waves are differentiable from a second echo created at a second tissue in response to the output acoustic waves, said first tissue having a thickness of less than 70 micrometers.

14. The medical device of claim 13 wherein the transducer is a component of an acoustic radiation force impulse imaging device.

15. The medical device of claim 13 wherein the transducer is a single transducer.

16. The medical device of claim 13 wherein the transducer has a center frequency of at least approximately 25 megahertz.

17. The medical device of claim 13 wherein the center frequency is between approximately 25 megahertz and 50 megahertz.

18. The medical device of claim 13 wherein a signal generator configured for interleaving diagnostic waveforms with push waveforms is in electrical communication with an amplifier and the transducer.

19. The medical device of claim 18 wherein the diagnostic waveforms comprise a voltage spike or monocycle.

20. The medical device of claim 18 wherein the push waveforms have a frequency approximately equal to the center frequency.

21. The medical device of claim 18 wherein the signal generator is configured to provide a sufficient number of push waveforms to effect a displacement in at least the first tissue layer.

22. The medical device of claim 18 wherein the signal generator is configured to provide a sufficient number of push waveforms to maintain the effected displacement.

23. The medical device of claim 13, wherein the first tissue is the epithelium and the second tissue is the stroma.

24. A system for noninvasively measuring the elasticity of ocular tissue, the system comprising:
the medical device of claim 1;
a digitizer;
a storage medium; and
a second device having a processor;
wherein the digitizer is configured to transform a first echo and a second echo into data,
wherein the storage medium is configured to store the data, and
wherein the processor is configured to calculate the properties.

25. The system of claim 24, wherein the second device is a PC, laptop, portable computing device, Smartphone or PDA.

26. The system of claim 25, further comprising a transmitting device.

27. The system of claim 26 wherein the transmitting device is configured to transmit data to the second device by wireless communication.

28. A method for determining biomechanical properties of a first tissue, the method comprising:
providing acoustic energy at a frequency of at least approximately 25 megahertz;
directing the energy toward the first tissue;
generating response pulses; and
determining a property of the first tissue,
wherein the acoustic energy is originated by a transducer,
wherein the response pulses are generated from echo waves received at the transducer that are reflected from the first tissue, and
wherein the material property is determined from the response pulses.

29. The method of claim 28 further comprising generating response pulses from echo waves received at the transducer that are reflected from a second tissue.

30. The method of claim 28 wherein the first tissue is the corneal epithelium and the second tissue is the corneal stroma.

31. The method of claim 28 further comprising digitizing the response pulses as data and storing the data.

32. The method of claim 29 further comprising calculating the viscosity of the first tissue and calculating the viscosity of the second tissue.

33. The method of claim 29 further comprising calculating the elasticity of the first tissue and calculating the elasticity of the second tissue.

34. The method of claim 29 further comprising assessing the cross-linking of the second tissue.

35. The method of claim 29 further comprising the step of driving the transducer with a series of diagnostic pulses interleaved with a series of push pulses.

36. The method of claim 35 further comprising the step of generating at least the push pulses at least approximately 25 megahertz.

37. The method of claim 35 further comprising the step of generating at least the push pulses at between approximately 25 megahertz and approximately 50 megahertz.

38. The method of claim 35 further comprising generating diagnostic acoustic waves and push acoustic waves.
